(19) United States
(12) Patent Application Publication
(10) Pub. No.: US 2011/0015135 A1
(43) Pub. Date: Jan. 20, 2011

(54) Antitumoral Treatments

(75) Inventors: Santiago Ramón Y Cajal Agüeras, Barcelona (ES); Javier Hernández Losa, Barcelona (ES); José Mª Jimeno Doñaque, Madrid (ES); Eric Raymond, Clichy (FR)

Correspondence Address: KING & SPALDING, 1185 AVENUE OF THE AMERICAS, NEW YORK, NY, 10036-4003, US

(73) Assignee: Pharma Mar S.A., Madrid (ES)

(21) Appl. No.: 12/920,433

(22) PCT Filed: Mar. 6, 2009
(86) PCT No.: PCT/EP09/52649

§ 371 Date: Aug. 31, 2010

(30) Foreign Application Priority Data
Mar. 7, 2008 (EP) 08380078.9

(51) Int. Cl. A61K 38/12; A61P 35/00; A61P 35/02

(52) U.S. Cl. 514/19.4; 514/19.9; 514/19.5; 514/19.6

(57) ABSTRACT

The present invention relates to combinations of PM02734 with another anticancer drug selected from Cisplatin, Gemcitabine, Paclitaxel, Oxaliplatin, 5-Fluorouracil, Trabectedin, Rapamycin, and Sunitinib, and the use of these combinations in the treatment of cancer.

### FIELD OF THE INVENTION

The present invention relates to the combination of PM02734 with other anticancer drugs, in particular the other anticancer drug is selected from Cisplatin, Gemcitabine, Paclitaxel, Oxaliplatin, 5-Fluorouracil, Trabectedin, Rapamycin, and Sunitinib, and the use of these combinations in the treatment of cancer.

### BACKGROUND OF THE INVENTION

Cancer develops when cells in a part of the body begin to grow out of control. Although there are many kinds of cancer, they all arise from out-of-control growth of abnormal cells. Cancer cells can invade nearby tissues and can spread through the bloodstream and lymphatic system to other parts of the body. There are several main types of cancer. Carcinoma is a malignant neoplasm, which is an uncontrolled and progressive abnormal growth, arising from epithelial cells. Epithelial cells cover internal and external surfaces of the body, including organs, lining of vessels, and other small cavities. Sarcoma is cancer arising from cells in bone, cartilage, fat, muscle, blood vessels, or other connective or supportive tissue. Leukemia is cancer that arises in blood-forming tissue such as the bone marrow, and causes large numbers of abnormal blood cells to be produced and enter the bloodstream. Lymphoma and multiple myeloma are cancers that arise from cells of the immune system.

In addition, cancer is invasive and tends to infiltrate the surrounding tissues and give rise to metastases. It can spread directly into surrounding tissues and also may be spread through the lymphatic and circulatory systems to other parts of the body.

Many treatments are available for cancer, including surgery and radiation for localised disease, and chemotherapy. However, the efficacy of available treatments for many cancer types is limited, and new, improved forms of treatment showing clinical benefits are needed. This is especially true for those patients presenting with advanced and/or metastatic disease and for patients relapsing with progressive disease after having been previously treated with established therapies which become ineffective or intolerable due to acquisition of resistance or to limitations in administration of the therapies due to associated toxicities.

Since the 1950s, significant advances have been made in the chemotherapeutic management of cancer. Unfortunately, more than 50% of all cancer patients either do not respond to initial therapy or experience relapse after an initial response to treatment and ultimately die from progressive metastatic disease. Thus, the ongoing commitment to the design and discovery of new anticancer agents is critically important.

Chemotherapy, in its classic form, has been focused primarily on killing rapidly proliferating cancer cells by targeting general cellular metabolic processes, including DNA, RNA, and protein biosynthesis. Chemotherapy drugs are divided into several groups based on how they affect specific chemical substances within cancer cells, which cellular activities or processes the drug interferes with, and which specific phases of the cell cycle the drug affects. The most commonly used types of chemotherapy drugs include: DNA-alkylating drugs (such as cyclophosphamide, ifosfamide, cisplatin, carboplatin, dacarbazine), antimetabolites (5-fluorouracil, capecitabine, 6-mercaptopurine, methotrexate, gemcitabine, cytarabine, fludarabine), mitotic inhibitors (such as paclitaxel, docetaxel, vinblastine, vincristine), anthracyclines (such as daunorubicin, doxorubicin, epirubicin, idarubicin, mitoxantrone), topoisomerase I and II inhibitors (such as topotecan, irinotecan, etoposide, teniposide), and hormone therapy (such as tamoxifen, flutamide).

The ideal antitumor drug would kill cancer cells selectively, with a wide index relative to its toxicity towards non-cancer cells and it would also retain its efficacy against cancer cells, even after prolonged exposure to the drug. Unfortunately, none of the current chemotherapies with these agents posses an ideal profile. Most posses very narrow therapeutic indexes and, in addition, cancerous cells exposed to slightly sublethal concentrations of a chemotherapeutic agent may develop resistance to such an agent, and quite often cross-resistance to several other antitumor agents.

PM02734 ((4S)-MeHex-D-Val-L-Thr-L-Val-D-Val-D-Pro-L-Orn-D-allo-Ile-cyclo(D-allo-Thr-D-allo-Ile-D-Val-L-Phe-Z-Dhb-L-Val)) is a novel synthetic depsipeptide related to the family of kahalalide compounds. This compound is the subject of WO 2004/035613 and has the following structure:

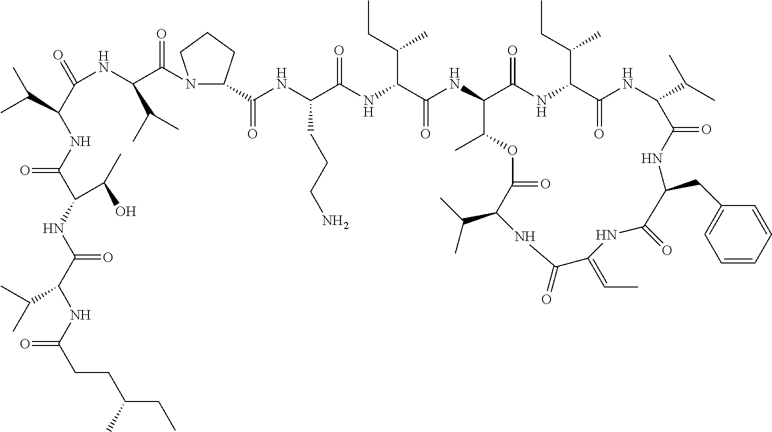

Kahalalide compounds are cyclic depsipeptides which were originally isolated from a Hawaiian herbivorous marine species of mollusk, Elysia rufescens, and its diet, the green alga Briopsis sp. Kahalalides A-G were described by Hamann et al. (J. Am. Chem. Soc. 1993, 115, 5825-5826 and J. Org. Chem. 1996, 61, 6594-6600) and many of them show activity against cancer and AIDS-related opportunistic infections. Some other natural kahalalide compounds have been also disclosed such as Kahalalide H and J by Scheuer et al. (J. Nat. Prod. 1997, 60, 562-567), Kahalalide O by Scheuer et al. (J. Nat. Prod. 2000, 63(1), 152-154), Kahalalide K by Kan et al. (J. Nat. Prod. 1999, 62(8), 1169-1172).

Of the kahalalide compounds of natural origin, Kahalalide F is the most promising because of its antitumoral activity. EP 610.078 reports that early preclinical in vitro screening studies identified micromolar activity of Kahalalide F against mouse leukemia (P388) and two human solid tumors: non-small cell lung (A549) and colon (HT-29). The primary mechanism of Kahalalide F action has not been identified yet, however it has been found that Kahalalide F is an NCI-COMPARE compound that induces sub G1 cell-cycle arrest and cytotoxicity independently of MDR, Her2, P53, and blc-2 (Janmaat et al. Proceedings of the 2^{nd} International Symposium on Signal Transduction Modulators in Cancer Therapy: 23-25 October, Amsterdam 2003: 60 (Abst. B02)). The COMPARE analysis in a panel of 60 human cancer cell lines genetically and molecularly characterized for cell proliferation pathways has included Kahalalide F in the list of new chemical entities that interact with the Erb/Her-neu pathway (Wosikowski et al. J. Natl. Cancer Inst. 1997, 89, 1505-1515). Sensitivity to Kahalalide F significantly correlated with baseline expression levels of ErbB3 (HER3), but not of other ErbB receptors, in a panel of established cell lines from different origins. Furthermore, the downstream P13K/Akt pathway coupled to ErbB3 receptor is also affected by Kahalalide F treatment. Kahalalide F decreases phosphorylated Akt levels and this reduction is associated with cytotoxicity in Kahalalide F-sensitive cell lines (Janmaat et al. Mol Pharmacol 2005, 68, 502-510).

PM02734 has showed significant improved efficacy in in vivo cancer models with respect to those activities observed with kahalalide compounds of natural origin, and specifically with Kahalalide F. PM02734 has demonstrated in vitro antitumor activity against a broad spectrum of tumor types such as leukemia, melanoma, breast, colon, ovary, pancreas, lung, and prostate, and has shown significant in vivo activity in xenografted murine models using human tumor cell types such as breast, prostate, and melanoma. Additionally, in PCT/US08/80309, PM02734 was evaluated in combination with EGFR tyrosine kinase inhibitors, specifically Erlotinib, for the treatment of lung cancer.

More information on PM02734 and other kahalalide compounds, in particular Kahalalide F and analogs thereof, their uses, formulations and synthesis can be found in the patent applications EP 610.078, WO 2004/035613, WO 01/58934, WO 2005/023846, WO 2004/075910, WO 03/033012, WO 02/36145, WO 2005/103072, and PCT/US08/80309. We incorporate by specific reference the content of each of these application texts.

Since cancer is a leading cause of death in animals and humans, several efforts have been and are still being undertaken in order to obtain a therapy active and safe to be administered to patients suffering from a cancer. The problem to be solved by the present invention is to provide anticancer therapies that are useful in the treatment of cancer.

### SUMMARY OF THE INVENTION

We have established that PM02734 potentiates other anticancer agents, in particular Cisplatin, Gemcitabine, Paclitaxel, Oxaliplatin, 5-Fluorouracil, Trabectedin, Rapamycin, and Sunitinib, and therefore they can be successfully used in combination therapy for the treatment of cancer.

Thus, this invention is directed to pharmaceutical compositions, kits, methods for the treatment of cancer using these combination therapies and uses of PM02734 in the manufacture of a medicament for combination therapy.

In accordance with one aspect of this invention, we provide effective combination therapies for the treatment of cancer based on PM02734 and using another anticancer drug selected from Cisplatin, Gemcitabine, Paclitaxel, Oxaliplatin, 5-Fluorouracil, Trabectedin, Rapamycin, and Sunitinib.

In another embodiment the invention encompasses a method of treating cancer comprising administering to a patient in need of such treatment a therapeutically effective amount of PM02734, or a pharmaceutically acceptable salt thereof, and a therapeutically effective amount of another anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, administered prior, during, or after administering PM02734. The two drugs may form part of the same composition, or be provided as a separate composition for administration at the same time or at a different time.

In another aspect the invention encompasses a method of increasing the therapeutic efficacy of an anticancer drug selected from Cisplatin, Gemcitabine, Paclitaxel, Oxaliplatin, 5-Fluorouracil, Trabectedin, Rapamycin, and Sunitinib in the treatment of cancer, which comprises administering to a patient in need thereof a therapeutically effective amount of PM02734, or a pharmaceutically acceptable salt thereof. PM02734 is administered prior, during, or after administering Cisplatin, Gemcitabine, Paclitaxel, Oxaliplatin, 5-Fluorouracil, Trabectedin, Rapamycin or Sunitinib.

In another embodiment the invention encompasses the use of PM02734, or a pharmaceutically acceptable salt thereof, for the manufacture of a medicament for the treatment of cancer, in combination therapy with another anticancer drug selected from Cisplatin, Gemcitabine, Paclitaxel, Oxaliplatin, 5-Fluorouracil, Trabectedin, Rapamycin, and Sunitinib.

In a related embodiment the invention encompasses the use of an anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, for the manufacture of a medicament for the treatment of cancer, in combination therapy with PM02734.

In a further aspect the invention encompasses a pharmaceutical composition comprising PM02734, or a pharmaceutically acceptable salt thereof, and/or another anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, to be used in combination therapy for the treatment of cancer.

The invention also encompasses a kit for use in the treatment of cancer which comprises a dosage form of PM02734, or a pharmaceutically acceptable salt thereof, and/or a dosage form of another anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, and instructions for the use of both drugs in combination.

In one preferred aspect, the present invention is concerned with synergistic combinations of PM02734, or a pharmaceutically acceptable salt thereof, with another anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof.

### BRIEF DESCRIPTION OF THE FIGURES

FIG. 1. Effects of the combination of PM02734 with Oxaliplatin in DU145 cell line. A) PM02734 administered prior to Oxaliplatin; B) Oxaliplatin administered prior to PM02734; C) PM02734 administered simultaneously with Oxaliplatin.

FIG. 2. Effects of the combination of PM02734 with Oxaliplatin in Colo205 cell line. A) PM02734 administered prior to Oxaliplatin; B) Oxaliplatin administered prior to PM02734; C) PM02734 administered simultaneously with Oxaliplatin.

FIG. 3. Effects of the combination of PM02734 with Cisplatin in DU145 cell line. A) PM02734 administered prior to Cisplatin; B) Cisplatin administered prior to PM02734; C) PM02734 administered simultaneously with Cisplatin.

FIG. 4. Effects of the combination of PM02734 with 5-FU in DU145 cell line. A) PM02734 administered prior to 5-FU; B) 5-FU administered prior to PM02734; C) PM02734 administered simultaneously with 5-FU.

FIG. 5. Effects of the combination of PM02734 with 5-FU in Colo205 cell line. A) PM02734 administered prior to 5-FU; B) 5-FU administered prior to PM02734; C) PM02734 administered simultaneously with 5-FU.

FIG. 6. Effects of the combination of PM02734 with Gemcitabine in DU145 cell line. A) PM02734 administered prior to Gemcitabine; B) Gemcitabine administered prior to PM02734; C) PM02734 administered simultaneously with Gemcitabine.

FIG. 7. Effects of the combination of PM02734 with Trabectedin in DU145 cell line. A) Trabectedin administered prior to PM02734; B) PM02734 administered simultaneously with Trabectedin.

FIG. 8. Effects of the combination of PM02734 with Rapamycin in DU145 cell line. A) PM02734 administered prior to Rapamycin; B) Rapamycin administered prior to PM02734; C) PM02734 administered simultaneously with Rapamycin.

FIG. 9. Effects of the combination of PM02734 with Rapamycin in Colo205 cell line. A) PM02734 administered prior to Rapamycin; B) Rapamycin administered prior to PM02734; C) PM02734 administered simultaneously with Rapamycin.

FIG. 10. Effects of the combination of PM02734 with Sunitinib in DU145 cell line. A) PM02734 administered prior to Sunitinib; B) Sunitinib administered prior to PM02734; C) PM02734 administered simultaneously with Sunitinib.

FIG. 11. Effects of the combination of PM02734 with Sunitinib in Colo205 cell line. A) PM02734 administered prior to Sunitinib; B) Sunitinib administered prior to PM02734.

### DETAILED DESCRIPTION OF THE INVENTION

We surprisingly found that the antitumor activity of Cisplatin, Gemcitabine, Paclitaxel, Oxaliplatin, 5-Fluorouracil, Trabectedin, Rapamycin, and Sunitinib is greatly enhanced in combination with PM02734. Thus, the present invention is directed to provide an efficacious treatment of cancer based on the combination of PM02734 with another anticancer drug selected from Cisplatin, Gemcitabine, Paclitaxel, Oxaliplatin, 5-Fluorouracil, Trabectedin, Rapamycin, and Sunitinib.

In the present application, by “cancer” it is meant to include tumors, neoplasias, and any other disease having as cause malignant tissue or cells.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The term “combination” as used throughout the specification, is meant to encompass the administration to a patient suffering form cancer of the referred therapeutic agents in the same or separate pharmaceutical formulations, and at the same time or at different times. If the therapeutic agents are administered at different times they should be administered sufficiently close in time to provide for the synergistic response to occur.

As mentioned above, PM02734 ((4S)-MeHex-D-Val-L-Thr-L-Val-D-Val-D-Pro-L-Orn-D-allo-Ile-cyclo(D-allo-Thr-D-allo-Ile-D-Val-L-Phe-Z-Dhb-L-Val)) is a synthetic depsipeptide with the following structure:

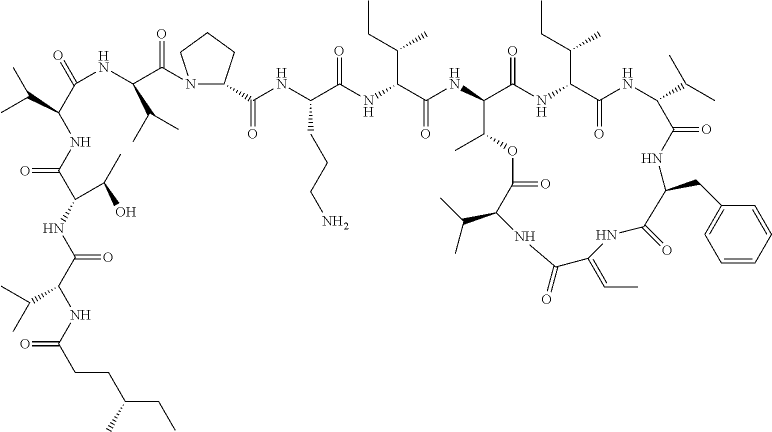

The term “PM02734” is intended here to cover any pharmaceutically acceptable salt, ester, solvate, hydrate, prodrug, or any other compound which, upon administration to the patient is capable of providing (directly or indirectly) the compound as described herein. The preparation of salts, esters, solvates, hydrates, prodrugs, and derivatives can be carried out by methods known in the art.

Any compound that is a prodrug of PM02734 is within the scope and spirit of the invention. The term “prodrug” is used in its broadest sense and encompasses those derivatives that are converted in vivo to PM02734. The prodrug can hydrolyze, oxidize, or otherwise react under biological conditions to provide PM02734. Such derivatives would readily occur to those skilled in the art, and include, for example, compounds where a free hydroxy group is converted into an ester derivative.

PM02734 for use in accordance of the present invention may be prepared following a synthetic process such as those disclosed in WO 2004/035613, WO 2005/103072, WO 01/58934, and WO 2005/023846, which are incorporated herein by reference.

Pharmaceutical compositions of PM02734, or of a pharmaceutically acceptable salt thereof, that can be used include solutions, suspensions, emulsions, lyophilized compositions, etc., with suitable excipients for intravenous administration. For further guidance on pharmaceutical compositions of PM02734, or a pharmaceutically acceptable salt thereof, see for example the formulations described in WO 2004/035613, which is incorporated herein by reference in its entirety.

Administration of PM02734, or a pharmaceutically acceptable salt thereof, or pharmaceutical compositions comprising the compound is preferably by intravenous infusion. Infusion times of up to 72 hours can be used, more preferably 1 to 24 hours, with either about 1 hour or about 3 hours most preferred. Short infusion times which allow treatment to be carried out without an overnight stay in hospital are especially desirable. However, infusion may be around 24 hours or even longer if required.

Preferably the administration of PM02734 is performed in cycles. In a preferred application method an intravenous infusion of PM02734 is given to the patients the first week of each cycle and the patients are allowed to recover for the remainder of the cycle. The preferred duration of each cycle is of either 1, 3, or 4 weeks. Multiple cycles can be given as needed. In an alternative dosing protocol, PM02734 is administered for say about 1 hour for 5 consecutive days every 3 or 4 weeks. Other protocols can be devised as variations. For further guidance on PM02734 administration and dosages, see for example WO 2004/035613 which is incorporated herein by reference.

Cisplatin is an inorganic platinum agent with the following structural formula:

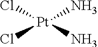

Cisplatin forms highly reactived, charged, platinum complexes which bind to nucleophilic groups such as GC-rich sites in DNA, inducing intrastrand and interstrand DNA cross-links, as well as DNA-protein cross-links. These cross-links result in apoptosis and cell growth inhibition. This drug is most commonly used to treat testicular, bladder, lung, gullet (oesophagus), stomach, and ovarian cancers. It is usually administered by intravenous infusion at a dose which depends on the schedule being used. Information about this drug is available on the extensive literature that exists on Cisplatin.

Gemcitabine is a nucleoside analogue with the following structural formula:

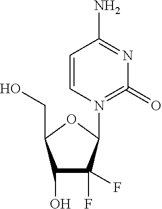

This drug is being marketed in the form of its hydrochloride salt with the trade name Gemzar®. This drug is currently indicated for the treatment of certain types of cancer, specifically for ovarian cancer, breast cancer, non-small cell lung cancer (NSCLC) and pancreatic cancer. As single agent, Gemcitabine is recommended to be administered by intravenous infusion at a dose of 1000 mg/m² over 30 minutes once weekly for up to 7 weeks, followed by a week of rest from treatment. Subsequent cycles should consist of infusions once weekly for 3 consecutive weeks out of every 4 weeks. Information about this drug is available on the website www.gemzar.com and the extensive literature on Gemcitabine.

Gemcitabine exhibits cell phase specificity, primarily killing cells undergoing DNA synthesis (S-phase) and also blocking the progression of cells through the G1/S-phase boundary. Gemcitabine is metabolized intracellularly by nucleoside kinases to the active diphosphate (dFdCDP) and triphosphate (dFdCTP) nucleosides. The cytotoxic effect of Gemcitabine is attributed to a combination of two actions of the diphosphate and the triphosphate nucleosides, which leads to inhibition of DNA synthesis. First, Gemcitabine diphosphate inhibits ribonucleotide reductase, which is responsible for catalyzing the reactions that generate the deoxynucleoside triphosphates for DNA synthesis. Inhibition of this enzyme by the diphosphate nucleoside causes a reduction in the concentrations of deoxynucleotides, including dCTP. Second, Gemcitabine triphosphate competes with dCTP for incorporation into DNA. The reduction in the intracellular concentration of dCTP (by the action of the diphosphate) enhances the incorporation of Gemcitabine triphosphate into DNA (self-potentiation). After the Gemcitabine nucleotide is incorporated into DNA, only one additional nucleotide is added to the growing DNA strands. After this addition, there is inhibition of further DNA synthesis. DNA polymerase epsilon is unable to remove the Gemcitabine nucleotide and repair the growing DNA strands (masked chain termination). In CEM T lymphoblastoid cells, Gemcitabine induces internucleosomal DNA fragmentation, one of the characteristics of programmed cell death.

Paclitaxel is a natural product with the following structural formula:

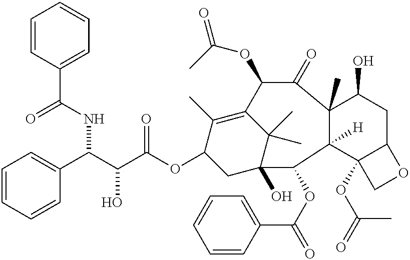

Paclitaxel (Taxol®) is a microtubule agent that promotes the assembly of microtubules from tubulin dimmers and stabilizes microtubules by preventing depolymerization. This stability results in the inhibition of the normal dynamic reorganization of the microtubule network that is essential for vital interphase and mitotic cellular functions. In addition, it induces abnormal arrays or “bundles” of microtubules throughout the cell cycle and multiple asters of microtubules during mitosis.

Paclitaxel is indicated for the treatment of ovarian, breast, and lung cancers, and AIDS-related Kaposi's sarcoma. It is usually administered by intravenous infusion at a dose which depends on the schedule being used. Information about this drug is available on the extensive literature that exists on Paclitaxel.

Sunitinib is a multi-kinase inhibitor with the following structural formula:

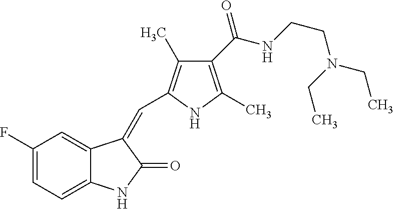

This drug is being marketed in the form of its malate salt with the trade name Sutent® and it is currently indicated for the treatment of certain types of cancer, specifically for gastrointestinal stromal tumor (GIST) and renal cell carcinoma. As single agent, the recommended dose is one 50 mg oral dose taken once daily, on a schedule of 4 weeks on treatment followed by 2 weeks off. Dose increase or reduction of 12.5 mg increments is recommended based on individual safety and tolerability. Information about this drug is available on the website www.sutent.com and the extensive literature on Sunitinib.

Oxaliplatin is a platinum-based chemotherapy drug in the same family as Cisplatin. Compared to Cisplatin the two amine groups are replaced by cyclohexyldiamine in Oxaliplatin for improved antitumor activity. In addition, the chlorine ligands of cisplatin are replaced by the oxalato bidentate derived from oxalic acid in order to improve water solubility.

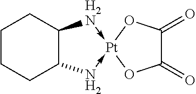

This drug is being marketed with the trade name Eloxatin® and it is typically administered in combination with 5-Fluorouracil and Leucovorin for the treatment of colorectal cancer. Information about this drug is available on the website www.eloxatin.com and the extensive literature on Oxaliplatin.

5-Fluorouracil (Fluorouracil, 5-FU) is a pyrimidine analog that belongs to the family of drugs called antimetabolites.

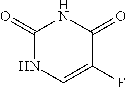

5-Fluorouracil has been in use in the treatment of cancer for about 40 years. Some of its principal use is in colorectal cancer and pancreatic cancer, in which it has been the established form of chemotherapy for decades. This drug acts in several ways, but principally as a thymidylate synthase inhibitor. Interrupting the action of this enzyme blocks synthesis of the pyrimidine thymidine, which is a nucleotide required for DNA replication. Like many anti-cancer drugs, 5-FU's effects are felt system wide but fall most heavily upon rapidly dividing cells that make heavy use of their nucleotide synthesis machinery, such as cancer cells. Further information about this drug is available on the extensive literature on 5-Fluorouracil.

Rapamycin, also known as sirolimus, is a macrolide first discovered as a product of the bacterium Streptomyces hygroscopicus.

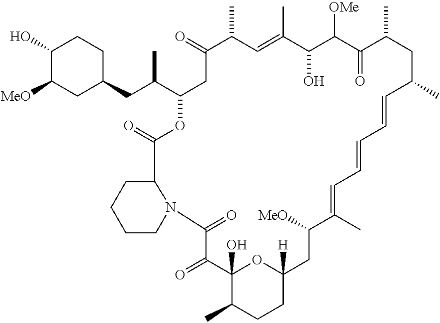

Rapamycin was originally developed as an antifungal agent. However, this was abandoned when it was discovered that it had potent immunosupressive and antiproliferative properties. The anti-proliferative effects of rapamycin may have a role in treating cancer. Information about this drug is available on the website www.rapamune.com and the extensive literature on Rapamycin.

Trabectedin, also known as ET-743, is a marine derived antitumoral agent which was first discovered in the tunicate Ecteinascidia turbinata.

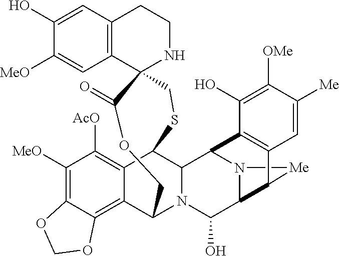

This drug is being marketed under the trade name Yondelis® for the treatment of soft tissue sarcoma. It is also undergoing clinical trials for the treatment of ovarian cancer, breast cancer, lung cancer, prostate cancer, and paediatric tumours. It binds to the minor groove of DNA and interferes with cell division and genetic transcription processes and DNA repair machinery. Information about this drug is available on the website www.yondelis.com and the extensive literature on Trabectedin.

Pharmaceutically acceptable salts of the drugs that are part of the combination of the invention referred to herein are synthesized from the parent compound, which contains a basic or acidic moiety, by conventional chemical methods. Generally, such salts are, for example, prepared by reacting the free acid or base forms of these compounds with a stoichiometric amount of the appropriate base or acid in water or in an organic solvent or in a mixture of the two. Generally, nonaqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methanesulphonate and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts.

In addition, any drug referred to herein may be in crystalline form either as free compound or as solvates (e.g. hydrates) and it is intended that both forms are within the scope of the present invention. Methods of solvation are generally known within the art.

Depending on the type of tumor and the development stage of the disease, anticancer effects of the methods of treatment of the present invention include, but are not limited to, inhibition of tumor growth, tumor growth delay, regression of tumor, shrinkage of tumor, increased time to regrowth of tumor on cessation of treatment, slowing of disease progression, and prevention of metastasis. It is expected that when a method of treatment of the present invention is administered to a patient, such as a human patient, in need of such treatment, said method of treatment will produce an effect, as measured by, for example, the extent of the anticancer effect, the response rate, the time to disease progression, or the survival rate. In particular, the methods of treatment of the invention are suited for human patients, especially those who are relapsing or refractory to previous chemotherapy. First line therapy is also envisaged.

The combination of the invention may be used alone or in combination with one or more of a variety of anti-cancer agents or supportive care agents.

In one embodiment, the invention relates to synergistic combinations employing PM02734, or a pharmaceutically acceptable salt thereof, and another anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof. An indication of synergy can be obtained by testing combinations and analyzing the results, for example by the Chou-Talalay method. Reference is made to Examples 1 to 4 to illustrate this point.

In another aspect, the invention is directed to the use of PM02734, or a pharmaceutically acceptable salt thereof, for the manufacture of a medicament for an effective treatment of cancer by combination therapy employing PM02734, or a pharmaceutically acceptable salt thereof, with another anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof.

In a related aspect, the invention is directed to the use of an anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, for the manufacture of a medicament for an effective treatment of cancer by combination therapy employing Cisplatin, Gemcitabine, Paclitaxel, Oxaliplatin, 5-Fluorouracil, Trabectedin, Rapamycin, or Sunitinib, or a pharmaceutically acceptable salt thereof, with PM02734, or a pharmaceutically acceptable salt thereof.

In a further aspect, the present invention is directed to a method of treating cancer comprising administering to a patient in need of such treatment a therapeutically effective amount of PM02734, or a pharmaceutically acceptable salt thereof, in combination with a therapeutically effective amount of another anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof.

The invention also provides a method of treating cancer comprising administering to a patient in need of such treatment a therapeutically effective amount of an anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, in combination with a therapeutically effective amount of PM02734, or a pharmaceutically acceptable salt thereof.

The combination drugs can be administered together, one after the other or separately in one combined unit dosage form or in two separate unit dosage forms. The unit dosage form may also be a fixed combination.

Simultaneous administration may, e.g., take place in the form of one fixed combination with two or more active ingredients, or by simultaneously administering two or more active ingredients that are formulated independently.

Sequential use administration preferably means administration of one (or more) components of a combination at one time point, other components at a different time point, that is, in a chronically staggered manner, preferably such that the combination shows more efficiency than the single compounds administered independently (especially showing synergism).

Separate use (administration) preferably means administration of the components of the combination independently of each other at different time points.

Thus, PM02734, or a pharmaceutically acceptable salt thereof, and the other anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, may be provided as separate medicaments for administration at the same time or at different times. Preferably, PM02734 and the other anticancer drug are provided as separate medicaments for administration at different times. When administered separately and at different times, either PM02734 or the other anticancer drug may be administered first. In addition, both drugs can be administered in the same day or at different days, and they can be administered using the same schedule or at different schedules during the treatment cycle. Thus, the pharmaceutical compositions of the present invention may comprise all the components (drugs) in a single pharmaceutically acceptable formulation. Alternatively, the components may be formulated separately and administered in combination with one another. Various pharmaceutically acceptable formulations well known to those of skill in the art can be used in the present invention. Additionally, the drugs of the combination may be given using different administration routes. For instance, one of the drugs may be in a form suitable for oral administration, for example as a tablet or capsule, and the other one in a form suitable for parenteral injection (including intravenous, subcutaneous, intramuscular, intravascular or infusion), for example as a sterile solution, suspension or emulsion. Alternatively, both drugs may be given by the same administration route. Selection of an appropriate formulation for use in the present invention can be performed routinely by those skilled in the art based upon the mode of administration and the solubility characteristics of the components of the composition.

The correct dosage of the compounds of the combination will vary according to the particular formulation, the mode of application, and the particular site, host and tumour being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

In another aspect, the present invention is directed to a kit for administering PM02734 in combination with another anticancer drug selected from Cisplatin, Gemcitabine, Paclitaxel, Oxaliplatin, 5-Fluorouracil, Trabectedin, Rapamycin, and Sunitinib in the treatment of cancer, comprising a supply of PM02734, or a pharmaceutically acceptable salt thereof, in dosage units for at least one cycle, and printed instructions for the use of both drugs in combination.

In a related aspect, the present invention is directed to a kit for administering an anticancer drug selected from Cisplatin, Gemcitabine, Paclitaxel, Oxaliplatin, 5-Fluorouracil, Trabectedin, Rapamycin, and Sunitinib in combination with PM02734 in the treatment of cancer, comprising a supply of the anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, in dosage units for at least one cycle, and printed instructions for the use of both drugs in combination.

In a related aspect, the present invention is directed to a kit for administering PM02734 in combination with another anticancer drug selected from Cisplatin, Gemcitabine, Paclitaxel, Oxaliplatin, 5-Fluorouracil, Trabectedin, Rapamycin, and Sunitinib in the treatment of cancer, comprising a supply of PM02734, or a pharmaceutically acceptable salt thereof, in dosage units for at least one cycle, a supply of an anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, in dosage units for at least one cycle, and printed instructions for the use of both drugs in combination.

In another aspect, the present invention also provides a pharmaceutical composition comprising PM02734, or a pharmaceutically acceptable salt thereof, and a pharmaceutically acceptable carrier, for use in combination with another anticancer drug selected from Cisplatin, Gemcitabine, Paclitaxel, Oxaliplatin, 5-Fluorouracil, Trabectedin, Rapamycin, and Sunitinib in the treatment of cancer.

In a further aspect, the present invention also provides a pharmaceutical composition comprising an anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, and a pharmaceutically acceptable carrier, for use in combination with PM02734 in the treatment of cancer.

In addition, the present invention also provides a pharmaceutical composition comprising PM02734, or a pharmaceutically acceptable salt thereof, an anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, and a pharmaceutically acceptable carrier, for use in the treatment of cancer.

In another aspect, the invention further provides for the use of PM02734, or a pharmaceutically acceptable salt thereof, in the preparation of a composition for use in combination with another anticancer drug selected from Cisplatin, Gemcitabine, Paclitaxel, Oxaliplatin, 5-Fluorouracil, Trabectedin, Rapamycin, and Sunitinib in the treatment of cancer.

In a related aspect, the invention further provides for the use of an anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, in the preparation of a composition for use in combination with PM02734 in the treatment of cancer.

And in a further aspect, the invention also provides for the use of PM02734, or a pharmaceutically acceptable salt thereof, and another anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, in the preparation of a composition for use in the treatment of cancer.

In another aspect, the invention further provides for the use of PM02734, or a pharmaceutically acceptable salt thereof, for the manufacture of a medicament for the treatment of cancer, in combination therapy with another anticancer drug selected from Cisplatin, Gemcitabine, Paclitaxel, Oxaliplatin, 5-Fluorouracil, Trabectedin, Rapamycin, and Sunitinib.

In a related aspect, the invention further provides for the use of an anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, for the manufacture of a medicament for the treatment of cancer, in combination therapy with PM02734.

In a related aspect, the invention further provides for the use of PM02734, or a pharmaceutically acceptable salt thereof, in combination with another anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, for the manufacture of a medicament for the treatment of cancer.

In another aspect, the invention further provides for the use of PM02734, or a pharmaceutically acceptable salt thereof, for the treatment of cancer, in combination therapy with another anticancer drug selected from Cisplatin, Gemcitabine, Paclitaxel, Oxaliplatin, 5-Fluorouracil, Trabectedin, Rapamycin, and Sunitinib.

In a related aspect, the invention further provides for the use of an anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, for the treatment of cancer, in combination therapy with PM02734.

In another aspect, the invention further provides for the use of PM02734, or a pharmaceutically acceptable salt thereof, in combination with another anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, for the treatment of cancer.

In another aspect, the invention further provides for the use of PM02734, or a pharmaceutically acceptable salt thereof, as a medicament, in combination therapy with another anticancer drug selected from Cisplatin, Gemcitabine, Paclitaxel, Oxaliplatin, 5-Fluorouracil, Trabectedin, Rapamycin, and Sunitinib.

In a related aspect, the invention further provides for the use of an anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, as a medicament, in combination therapy with PM02734.

In another aspect, the invention further provides for the use of PM02734, or a pharmaceutically acceptable salt thereof, in combination with another anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, as a medicament.

In another aspect, the invention further provides for the use of PM02734, or a pharmaceutically acceptable salt thereof, as a medicament for the treatment of cancer, in combination therapy with another anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof.

In a related aspect, the invention further provides for the use of an anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, as a medicament for the treatment of cancer, in combination therapy with PM02734, or a pharmaceutically acceptable salt thereof.

In another aspect, the invention further provides for the use of PM02734, or a pharmaceutically acceptable salt thereof, in combination with another anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, as a medicament for the treatment of cancer.

In another aspect, the invention provides PM02734, or a pharmaceutically acceptable salt thereof, for the treatment of cancer comprising administering a therapeutically effective amount of PM02734, or a pharmaceutically acceptable salt thereof, in combination with a therapeutically effective amount of an anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof.

In a related aspect, the invention further provides an anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, for the treatment of cancer comprising administering a therapeutically effective amount of an anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, in combination with a therapeutically effective amount of PM02734, or a pharmaceutically acceptable salt thereof.

In another aspect, the invention provides for the treatment of cancer comprising the administration of a therapeutically effective amount of PM02734, or pharmaceutically acceptable salt thereof, in combination with the administration of a therapeutically effective amount of another anticancer drug selected from Cisplatin, Gemcitabine, Paclitaxel, Oxaliplatin, 5-Fluorouracil, Trabectedin, Rapamycin, and Sunitinib, or a pharmaceutically salt thereof, wherein the combination may be administered together or separately.

Preferably, the combination of PM02734, or a pharmaceutically acceptable salt thereof, with another anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, is used for the treatment of testicular cancer, bladder cancer, lung cancer, gullet cancer, stomach cancer, ovarian cancer, breast cancer, pancreatic cancer, colorectal cancer (also known as colon cancer), leukemia, melanoma, and prostate cancer. Specially preferred is the use of the combination for the treatment of lung cancer, breast cancer, colorectal cancer, and prostate cancer.

In one embodiment, cancer cells are contacted, or otherwise treated, with a combination of PM02734, or a pharmaceutically acceptable salt thereof, and another anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof. The cancer cells are preferably human and include carcinoma cells, sarcoma cells, leukemia cells, lymphoma cells and myeloma cells. More preferably, the cancer cells include testicular cancer cells, bladder cancer cells, lung cancer cells, gullet cancer cells, stomach cancer cells, ovarian cancer cells, breast cancer cells, pancreatic cancer cells, colorectal cancer cells, leukemia cells, melanoma cells, and prostate cancer cells. In particular, the cancer cells include human lung cancer cells, human breast cancer cells, human colorectal cancer cells, and human prostate cancer cells. In addition, the combination provides a synergistic inhibitory effect against cancer cells, particularly against human lung cancer cells, human breast cancer cells, human colorectal cancer cells, and human prostate cancer cells.

For example, the combination inhibits proliferation or survival of contacted cancer cells. A lower level of proliferation or survival of the contacted cancer cells compared to the non-contacted cancer cells supports the combination of PM02734, or a pharmaceutically acceptable salt thereof, and another anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, as being effective for treating a patient with that particular type of cancer.

In another aspect, the invention provides for a method for inhibiting the growth of cancer cells comprising contacting said cancer cells with an effective amount of PM02734, or a pharmaceutically acceptable salt thereof, in combination with another anticancer drug selected from Cisplatin, Gemcitabine, Paclitaxel, Oxaliplatin, 5-Fluorouracil, Trabectedin, Rapamycin, and Sunitinib.

In a related aspect, the invention provides for a method for inhibiting the growth of cancer cells comprising contacting said cancer cells with an effective amount of an anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, in combination with PM02734.

In a related aspect, the invention provides for a method for inhibiting the growth of cancer cells comprising contacting said cancer cells with an effective combination of PM02734, or a pharmaceutically acceptable salt thereof, and an anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, together or separately.

In another aspect, the invention provides for a method for inhibiting the growth of cancer cells comprising contacting said cancer cells with a synergistic combination of PM02734, or a pharmaceutically acceptable salt thereof, and another anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, together or separately, wherein said combination provides improved inhibition against cancer cell growth as compared to (i) PM02734, or a pharmaceutically acceptable salt thereof, in the absence of an anticancer drug selected from Cisplatin, Gemcitabine, Paclitaxel, Oxaliplatin, 5-Fluorouracil, Trabectedin, Rapamycin, and Sunitinib, or (ii) an anticancer drug selected from Cisplatin, Gemcitabine, Paclitaxel, Oxaliplatin, 5-Fluorouracil, Trabectedin, Rapamycin, and Sunitinib, or pharmaceutically acceptable salt thereof, in the absence of PM02734.

In another aspect, the invention provides for a pharmaceutical composition comprising an effective amount of PM02734, or a pharmaceutically acceptable salt thereof, for use in combination with another anticancer drug selected from Cisplatin, Gemcitabine, Paclitaxel, Oxaliplatin, 5-Fluorouracil, Trabectedin, Rapamycin, and Sunitinib for inhibiting the growth of cancer cells.

In a related aspect, the invention provides for a pharmaceutical composition comprising an effective amount of an anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, for use in combination with PM02734 for inhibiting the growth of cancer cells.

In a related aspect, the invention provides for a pharmaceutical composition comprising an effective combination of PM02734, or a pharmaceutically acceptable salt thereof, and another anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, for inhibiting the growth of cancer cells.

In another aspect, the invention provides for a pharmaceutical composition comprising a synergistic combination of PM02734, or a pharmaceutically acceptable salt thereof, and another anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, for inhibiting the growth of cancer cells, wherein said combination provides improved inhibition against cancer cell growth as compared to (i) PM02734, or a pharmaceutically acceptable salt thereof, in the absence of an anticancer drug selected from Cisplatin, Gemcitabine, Paclitaxel, Oxaliplatin, 5-Fluorouracil, Trabectedin, Rapamycin, and Sunitinib, or (ii) an anticancer drug selected from Cisplatin, Gemcitabine, Paclitaxel, Oxaliplatin, 5-Fluorouracil, Trabectedin, Rapamycin, and Sunitinib, or pharmaceutically acceptable salt thereof, in the absence of PM02734.

The following examples further illustrate the invention. These examples should not be interpreted as a limitation of the scope of the invention.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

### Examples

### Example 1

### In Vitro Studies to Determine the Effect of PM02734 in Combination with Cisplatin, Gemcitabine, and Paclitaxel on Lung Cancer Cell Lines

PM02734 as a single agent or in combination with another anticancer drug selected from Cisplatin, Gemcitabine, and Paclitaxel was evaluated against several tumor cell lines related with lung carcinoma. Specifically, the cell lines tested were A549 which was obtained from ATCC (ATCC no. CCL-185), DV90 which was obtained from the European Collection DSMZ (ACC no. 307) (www.dsmz.de), and HOP62 which obtained from the American collection DTP (Developmental Therapeutics Program of the NCI) (http://dtp.nci.nih.gov/index.html). These cell lines were cultured in the following media:
A549 cells were grown in HAMS F12 medium, supplemented with 2 mM L-Glutamine and 1.5 g/l sodium bicarbonate
DV90 and HOP62 cells were grown in RPMI medium, supplemented with 2 mM L-Glutamine.

All culture media were supplemented with 10% Foetal Bovine Serum (FBS), 100 μg/ml penicillin, 100 μg/ml streptomycin, 0.25 μg/ml amphotericin B and 25 mM HEPES.

The screening was performed in two parts:

a. In the first set of assays, IC₅₀ values were determined for each drug after 72 hours of drug exposure in each of the tumor cell lines.

Cells were plated at 70% confluence in 24-well plates. In particular, 40000 cells/well for A549 cell line, 75000 cells/well for DV90 cell line and 60000 cells/well for HOP62 cell line were plated. After 24 h the cells were exposed to different concentrations of PM02734 trifluoroacetate salt, Cisplatin, Paclitaxel or Gemcitabine hydrochloride for 72 h at 37° C., 5% CO₂ and humidity levels higher than 80%. At the end of the incubation period viability was assayed by the crystal violet method. Briefly, cells were washed with PBS, then fixed in glutaraldehyde 2% for 20 min, washed again twice in PBS and stained with crystal violet for 20 min, and then washed with abundant deionized water. Colorant was recovered with 1% acetic acid and Optical Density was evaluated at 590 nm. Cell viability was correlated to the amount of colorant quantified sprectrophotometrically at 590 nm. Experiments were performed in triplicate.

The individual IC₅₀ values obtained for each of the drugs are shown in table I.

TABLE I

| | IC₅₀ (M) | IC₅₀ (M) | IC₅₀ (M) | IC₅₀ (M) |
|---|---|---|---|---|
| Cell line | PM02734 | Cisplatin | Paclitaxel | Gemcitabine |
| A549 | 2E−07 | 8.25E−06 | 7E−09 | 4E−09 |
| DV90 | 3E−07 | 16.5E−06 | 3E−08 | 4E−07 |
| HOP62 | 4E−06 | 8.25E−06 | 4E−08 | 7.5E−7 |

b. In a second set of assays, each cell line was incubated with PM02734 in combination with each of the above mentioned anticancer agents.

PM02734 trifluoroacetate salt was combined with Gemcitabine hydrochloride salt, Cisplatin or Paclitaxel, at a fixed ratio of doses that corresponded to 0.125, 0.25, 0.5, 1 and 2 times the individual IC₅₀ values for each drug alone. As exception, when PM02734 trifluoroacetate salt was combined with Gemcitabine hydrochloride salt or Paclitaxel in DV90 cell line the fixed ratio of doses tested corresponded to 0.0625, 0.125, 0.25, 0.5, and 1 times the individual IC₅₀ values for each drug alone.

The combination index (CI) was calculated based on the Chou-Talalay equation, which takes into account both potency and the shape of the dose-effect curve. CI<1, CI=1, CI>1 indicate synergism, additive effect, and antagonism, respectively (Chou T C and Talalay P. Adv. Enzyme Regul. 1984, 22, 27-55). CalcuSyn software (Biosoft, Ferguson, Mo.) was used for the Chou-Talalay combination index analysis.

Table II provides the Combination Index (CI) that was obtained when combining PM02734 with Cisplatin at different doses on A549 cell line.

TABLE II

| PM02734 (M) | Cisplatin (M) | CI |
|---|---|---|
| 2.5E−08 | 1.03E−06 | 4.899 |
| 5E−08 | 2.06E−06 | 1.148 |
| 1E−07 | 4.12E−06 | 1.069 |
| 2E−07 | 8.25E−06 | 0.752 |
| 4E−07 | 16.5E−06 | 1.217 |

Table III provides the Combination Index (CI) that was obtained when combining PM02734 with Cisplatin at different doses on DV90 cell line.

TABLE III

| PM02734 (M) | Cisplatin (M) | CI |
|---|---|---|
| 3.75E−08 | 2.06E−06 | 1.325 |
| 7.5E−08 | 4.12E−06 | 1.030 |
| 1.5E−07 | 8.25E−06 | 1.405 |
| 3E−07 | 16.5E−06 | 0.482 |
| 6E−07 | 33E−06 | 0.888 |

Table IV provides the Combination Index (CI) that was obtained when combining PM02734 with Cisplatin at different doses on HOP62 cell line.

TABLE IV

| PM02734 (M) | Cisplatin (M) | CI |
|---|---|---|
| 5E−07 | 1.03E−06 | 38.881 |
| 1E−06 | 2.06E−06 | 2.129 |
| 2E−06 | 4.12E−06 | 3.640 |
| 4E−06 | 8.25E−06 | 0.850 |
| 8E−06 | 16.5E−06 | 0.751 |

According to these assays, it was found that the combination of PM02734 with Cisplatin showed synergism at high doses of both drugs in lung carcinoma cell lines, specially in DV90 and HOP62 cell lines.

Table V provides the Combination Index (CI) that was obtained when combining PM02734 with Paclitaxel at different doses on A549 cell line.

TABLE V

| PM02734 (M) | Paclitaxel (M) | CI |
|---|---|---|
| 2.5E−08 | 8.75E−10 | 0.780 |
| 5E−08 | 1.75E−09 | 0.816 |
| 1E−07 | 3.5E−09 | 0.939 |
| 2E−07 | 7E−09 | 1.355 |
| 4E−07 | 1.4E−08 | 1.657 |

Table VI provides the Combination Index (CI) that was obtained when combining PM02734 with Paclitaxel at different doses on DV90 cell line.

TABLE VI

| PM02734 (M) | Paclitaxel (M) | CI |
|---|---|---|
| 1.87E−08 | 1.87E−10 | 0.736 |
| 3.75E−08 | 3.75E−10 | 0.851 |
| 7.5E−08 | 7.5E−10 | 0.927 |
| 1.5E−07 | 1.5E−09 | 1.130 |
| 3E−07 | 3E−09 | 1.739 |

Table VII provides the Combination Index (CI) that was obtained when combining PM02734 with Paclitaxel at different doses on HOP62 cell line.

TABLE VII

| PM02734 (M) | Paclitaxel (M) | CI |
|---|---|---|
| 5E−07 | 5E−09 | 0.417 |
| 1E−06 | 1E−08 | 0.399 |
| 2E−06 | 2E−08 | 0.633 |
| 4E−06 | 4E−08 | 0.860 |
| 8E−06 | 8E−08 | 1.032 |

According to these assays, it was found that the combination of PM02734 with Paclitaxel showed synergism in lung carcinoma cell lines. Specially, synergism was observed at low doses of both drugs in A549 and DV90 cell lines and at a broad range of doses in HOP62 cell lines.

Table VIII provides the Combination Index (CI) that was obtained when combining PM02734 with Gemcitabine at different doses on A549 cell line.

TABLE VIII

| PM02734 (M) | Gemcitabine (M) | CI |
|---|---|---|
| 2.5E−08 | 5E−10 | 0.423 |
| 5E−08 | 1E−09 | 0.630 |
| 1E−07 | 2E−09 | 0.857 |
| 2E−07 | 4E−09 | 1.200 |
| 4E−07 | 8E−09 | 1.159 |

Table IX provides the Combination Index (CI) that was obtained when combining PM02734 with Gemcitabine at different doses on DV90 cell line.

TABLE IX

| PM02734 (M) | Gemcitabine (M) | CI |
|---|---|---|
| 1.87E−08 | 2.5E−08 | 0.417 |
| 3.75E−08 | 5E−08 | 2.460 |
| 7.5E−08 | 1E−07 | 2.815 |
| 1.5E−07 | 2E−07 | 0.622 |
| 3E−07 | 4E−07 | 0.814 |

Table X provides the Combination Index (CI) that was obtained when combining PM02734 with Gemcitabine at different doses on HOP62 cell line.

TABLE X

| PM02734 (M) | Gemcitabine (M) | CI |
|---|---|---|
| 5E−07 | 9.37E−08 | 1.178 |
| 1E−06 | 1.87E−07 | 0.803 |
| 2E−06 | 3.75E−07 | 0.682 |
| 4E−06 | 7.5E−07 | 1.290 |
| 8E−06 | 1.5E−06 | 1.589 |

According to these assays, it was found that the combination of PM02734 with Gemcitabine showed synergism in lung carcinoma cell lines. Specially, synergism was observed at low doses of both drugs in A549 and HOP62 cell lines and at low and high doses in DV90 cell line.

### Example 2

### In Vitro Studies to Determine the Effect of PM02734 in Combination with Cisplatin, Gemcitabine, and Paclitaxel on Breast Cancer Cell Lines

PM02734 trifluoroacetate salt as a single agent or in combination with another anticancer drug selected from Cisplatin, Gemcitabine hydrochloride, and Paclitaxel was evaluated against several tumor cell lines related with breast adenocarcinoma. Specifically, the cell lines tested were MDA-MB-231 (ATCC no. HTB-26), MDA-MB-435 (ATCC no. HTB-129), and MCF7 (ATCC no. HTB-22) which were all obtained from ATCC. These cell lines were cultured in the following media:
MDA-MB-231 and MDA-MB-435 cells were grown in DMEM, supplemented with 2 mM L-Glutamine and 4.5 g/l glucose.
MCF-7 cells were grown in RPMI medium, supplemented with 2 mM L-Glutamine.

All culture media were supplemented with 10% Foetal Bovine Serum (FBS), 100 μg/ml penicillin, 100 μg/ml streptomycin, 0.25 μg/ml amphotericin B and 25 mM HEPES.

The screening was performed in two parts:

a. In the first set of assays, ICₛₒ values were determined for each drug after 72 hours of drug exposure in each of the tumor cell lines.

Cells were plated at 75% confluence in 24-well plates. In particular, 30000 cells/well for MDA-MB-231 cell line, 40000 cells/well for MDA-MB-435 cell line and 60000 cells/well for MCF-7 cell line were plated. Following the same methodology as those disclosed in Example 1 was used.

The individual IC₅₀ values are shown in table XI.

TABLE XI

| | IC₅₀ (M) | IC₅₀ (M) | IC₅₀ (M) | IC₅₀ (M) |
|---|---|---|---|---|
| Cell line | PM02734 | Cisplatin | Paclitaxel | Gemcitabine |
| MDA-MB-231 | 6.5E−06 | 8.25E−06 | 2.5E−08 | 1E−06 |
| MDA-MB-435 | 5E−07 | 2.64E−06 | 2.5E−08 | 4E−08 |
| MCF7 | 6E−07 | 16.5E−06 | 7.5E−09 | 9E−7 |

b. In a second set of assays, each cell line was incubated with PM02734 trifluoroacetate salt in combination with each of the above mentioned anticancer agents. The same methodology as those disclosed in Example 1 was used.

Table XII provides the Combination Index (CI) that was obtained when combining PM02734 with Cisplatin at different doses on MDA-MB-231 cell line.

TABLE XII

| PM02734 (M) | Cisplatin (M) | CI |
|---|---|---|
| 8.12E−07 | 1.03E−06 | 0.723 |
| 1.62E−06 | 2.06E−06 | 0.608 |
| 3.25E−06 | 4.12E−06 | 0.660 |
| 6.5E−06 | 8.25E−06 | 0.917 |
| 13E−06 | 16.5E−06 | 1.605 |

Table XIII provides the Combination Index (CI) that was obtained when combining PM02734 with Cisplatin at different doses on MDA-MB-435 cell line.

TABLE XIII

| PM02734 (M) | Cisplatin (M) | CI |
|---|---|---|
| 6.25E−08 | 3.3−E07 | 1.422 |
| 1.25E−07 | 6.6−E07 | 0.725 |
| 2.5E−07 | 1.32−E06 | 1.281 |
| 5E−07 | 2.64−E06 | 2.284 |
| 1E−06 | 5.28−E06 | 0.667 |

Table XIV provides the Combination Index (CI) that was obtained when combining PM02734 with Cisplatin at different doses on MCF7 cell line.

TABLE XIV

| PM02734 (M) | Cisplatin (M) | CI |
|---|---|---|
| 7.5E−08 | 2.06E−06 | 1.827 |
| 1.5E−07 | 4.12E−06 | 1.395 |
| 3E−07 | 8.25E−06 | 1.324 |
| 6E−07 | 16.5E−06 | 3.458 |
| 1.2E−06 | 33E−06 | 0.720 |

According to these assays, it was found that the combination of PM02734 with Cisplatin showed synergism in breast carcinoma cell lines. Specially, synergism was observed at low doses of both drugs in MDA-MB-231 cell line, at low and high doses in MDA-MB-435 cell line, and at high doses in MCF7 cell line.

Table XV provides the Combination Index (CI) that was obtained when combining PM02734 with Paclitaxel at different doses on MDA-MB-231 cell line.

TABLE XV

| PM02734 (M) | Paclitaxel (M) | CI |
|---|---|---|
| 8.12E−07 | 3.12E−09 | 15.149 |
| 1.62E−06 | 6.25E−09 | 0.926 |
| 3.25E−06 | 1.25E−08 | 1.175 |
| 6.5E−06 | 2.5E−08 | 2.272 |
| 13E−06 | 5E−08 | 2.818 |

Table XVI provides the Combination Index (CI) that was obtained when combining PM02734 with Paclitaxel at different doses on MDA-MB-435 cell line.

TABLE XVI

| PM02734 (M) | Paclitaxel (M) | CI |
|---|---|---|
| 6.25E−08 | 3.12E−09 | 0.736 |
| 1.25E−07 | 6.25E−09 | 0.726 |
| 2.5E−07 | 1.25E−08 | 1.355 |
| 5E−07 | 2.5E−08 | 2.367 |
| 1E−06 | 5E−08 | 0.784 |

Table XVII provides the Combination Index (CI) that was obtained when combining PM02734 with Paclitaxel at different doses on MCF7 cell line.

TABLE XVII

| PM02734 (M) | Paclitaxel (M) | CI |
|---|---|---|
| 7.5E−08 | 9.34E−10 | 0.817 |
| 1.5E−07 | 1.87E−09 | 1.289 |
| 3E−07 | 3.75E−09 | 1.393 |
| 6E−07 | 7.5E−09 | 1.058 |
| 1.2E−06 | 1.5E−08 | 1.141 |

According to these assays, it was found that the combination of PM02734 with Paclitaxel showed synergism in breast carcinoma cell lines. Specially, synergism was observed at low and high doses of both drugs in MDA-MB-435 cell line, and at low doses in MCF7 cell line.

Table XVIII provides the Combination Index (CI) that was obtained when combining PM02734 with Gemcitabine at different doses on MDA-MB-231 cell line.

TABLE XVIII

| PM02734 (M) | Gemcitabine (M) | CI |
|---|---|---|
| 8.12E−07 | 1.25E−07 | 0.170 |
| 1.62E−06 | 2.5E−07 | 0.243 |
| 3.25E−06 | 5E−07 | 0.434 |
| 6.5E−06 | 1E−06 | 0.780 |
| 13E−06 | 2E−06 | 1.170 |

Table XIX provides the Combination Index (CI) that was obtained when combining PM02734 with Gemcitabine at different doses on MDA-MB-435 cell line.

TABLE XIX

| PM02734 (M) | Gemcitabine (M) | CI |
|---|---|---|
| 6.25E−08 | 5E−09 | 1.698 |
| 1.25E−07 | 1E−08 | 0.609 |
| 2.5E−07 | 2E−08 | 0.640 |
| 5E−07 | 4E−08 | 0.940 |
| 1E−06 | 8E−08 | 1.192 |

Table XX provides the Combination Index (CI) that was obtained when combining PM02734 with Gemcitabine at different doses on MCF7 cell line.

TABLE XX

| PM02734 (M) | Gemcitabine (M) | CI |
|---|---|---|
| 7.5E−08 | 1.12E−07 | 9.273 |
| 1.5E−07 | 2.25E−07 | 1.712 |
| 3E−07 | 4.5E−07 | 1.466 |
| 6E−07 | 9E−07 | 0.897 |
| 1.2E−06 | 1.8E−06 | 0.769 |

According to these assays, it was found that the combination of PM02734 with Gemcitabine showed synergism in breast carcinoma cell lines. Specially, synergism was observed at a broad range of doses range of both drugs in MDA-MB-231 and MDA-MB-435 cell lines, and at high doses of both drugs in MCF-7 cell line.

### Example 3

### In Vitro Studies to Determine the Effect of PM02734 in Combination with Cisplatin, Gemcitabine, and Paclitaxel on Colon Cancer Cell Lines

PM02734 trifluoroacetate salt as a single agent or in combination with another anticancer drug selected from Cisplatin, Gemcitabine hydrochloride, and Paclitaxel was evaluated against several tumor cell lines related with colorectal adenocarcinoma. Specifically, the cell lines tested were DLD1 (ATCC no. CCL-221) and HT29 (ATCC no. HTB-38) which were obtained from ATCC. These cell lines were cultured in the following media:
DLD1 cells were grown in DMEM, supplemented with 2 mM L-Glutamine and 4.5 g/l glucose.
HT29 cells were grown in RPMI medium, supplemented with 2 mM L-Glutamine.

All culture media were supplemented with 10% Foetal Bovine Serum (FBS), 100 μg/ml penicillin, 100 μg/ml streptomycin, 0.25 μg/ml amphotericin B and 25 mM HEPES.

The screening was performed in two parts:

a. In the first set of assays, IC₅₀ values were determined for each drug after 72 hours of drug exposure in each of the tumor cell lines.

Cells were plated at 75% confluence in 24-well plates. In particular, 60000 cells/well for DLD1 cell line and 75000 cells/well for HT29 cell line were plated in 24-well plates. Following the same methodology as those disclosed in Example 1 was used.

The individual IC₅₀ values are shown in table XXI.

TABLE XXI

| | IC₅₀ (M) | IC₅₀ (M) | IC₅₀ (M) | IC₅₀ (M) |
|---|---|---|---|---|
| Cell line | PM02734 | Cisplatin | Paclitaxel | Gemcitabine |
| DLD1 | 3.5E−07 | 24.75E−06 | 4E−08 | 2E−06 |
| HT29 | 5.5E−07 | 6.6E−06 | 1E−08 | 5.5E−07 |

b. In a second set of assays, each cell line was incubated with PM02734 trifluoroacetate salt in combination with each of the above mentioned anticancer agents. The same methodology as those disclosed in Example 1 was used.

Table XXII provides the Combination Index (CI) that was obtained when combining PM02734 with Cisplatin at different doses on DLD 1 cell line.

TABLE XXII

| PM02734 (M) | Cisplatin (M) | CI |
|---|---|---|
| 4.37E−08 | 3.09E−06 | 0.633 |
| 8.75E−08 | 6.19E−06 | 0.792 |
| 1.75E−07 | 12.37E−06 | 1.105 |
| 3.5E−07 | 24.75E−06 | 0.607 |
| 7E−07 | 49.5E−06 | 0.821 |

Table XXIII provides the Combination Index (CI) that was obtained when combining PM02734 with Cisplatin at different doses on HT29 cell line.

Table XXIII

| PM02734 (M) | Cisplatin (M) | CI |
|---|---|---|
| 6.87E−08 | 8.25E−07 | 1.762 |
| 1.37E−07 | 1.65E−06 | 1.225 |
| 2.75E−07 | 3.3E−06 | 1.285 |
| 5.5E−07 | 6.6E−06 | 1.972 |
| 1.1E−06 | 13.2E−06 | 1.102 |

According to these assays, it was found that the combination of PM02734 with Cisplatin showed synergism in colorectal adenocarcinoma cell lines. Specially, synergism was observed at a broad range of doses of both drugs in DLD 1 cell line.

Table XXIV provides the Combination Index (CI) that was obtained when combining PM02734 with Paclitaxel at different doses on DLD1 cell lines.

Table XXIV

| PM02734 (M) | Paclitaxel (M) | CI |
|---|---|---|
| 4.37E−08 | 5E−09 | 0.554 |
| 8.75E−08 | 1E−08 | 0.754 |
| 1.75E−07 | 2E−08 | 1.420 |
| 3.5E−07 | 4E−08 | 2.542 |
| 7E−07 | 8E−08 | 0.899 |

Table XXV provides the Combination Index (CI) that was obtained when combining PM02734 with Paclitaxel at different doses on HT29 cell lines.

TABLE XXV

| PM02734 (M) | Paclitaxel (M) | CI |
|---|---|---|
| 6.87E−08 | 1.25E−09 | 0.948 |
| 1.37E−07 | 2.5E−09 | 1.113 |
| 2.75E−07 | 5E−09 | 1.333 |
| 5.5E−07 | 1E−08 | 0.931 |
| 1.1E−06 | 2E−08 | 1.183 |

According to these assays, it was found that the combination of PM02734 with Paclitaxel showed synergism in colorectal adenocarcinoma cell lines. Specially, synergism was observed at a broad range of doses of both drugs in DLD1 cell line.

Table XXVI provides the Combination Index (CI) that was obtained when combining PM02734 with Gemcitabine at different doses on DLD 1 cell lines.

TABLE XXVI

| PM02734 (M) | Gemcitabine (M) | CI |
|---|---|---|
| 4.37E−08 | 2.5E−07 | 1.590 |
| 8.75E−08 | 5E−07 | 0.572 |
| 1.75E−07 | 1E−06 | 0.626 |
| 3.5E−07 | 2E−06 | 0.908 |
| 7E−07 | 4E−06 | 1.171 |

Table XXVII provides the Combination Index (CI) that was obtained when combining PM02734 with Gemcitabine at different doses on HT29 cell lines.

TABLE XXVII

| PM02734 (M) | Gemcitabine (M) | CI |
|---|---|---|
| 6.87E−08 | 6.87E−08 | 5.061 |
| 1.37E−07 | 1.37E−07 | 1.601 |
| 2.75E−07 | 2.75E−07 | 1.320 |
| 5.5E−07 | 5.5E−07 | 1.128 |
| 1.1E−06 | 1.1E−06 | 0.914 |

According to these assays, it was found that the combination of PM02734 with Gemcitabine showed synergism in colorectal adenocarcinoma cell lines. Specially, synergism was observed at a broad range of doses of both drugs in DLD 1 cell line.

### Example 4

### In Vitro Studies to Determine the Effect of PM02734 in Combination with Oxaliplatin, Cisplatin, 5-FU, Gemcitabine, Trabectedin, Rapamycin, and Sunitinib on Prostate and Colon Cancer Cell Lines

PM02734 in combination with another anticancer drug selected from Oxaliplatin, Cisplatin, 5-FU, Gemcitabine, Trabectedin, Rapamycin, and Sunitinib was evaluated against two tumor cell lines, one related to prostate cancer and the other one to colon cancer. Specifically, the cell lines tested were DU145 (prostate cancer) and Colo205 (colon cancer) which were obtained from the ATCC (Rockville, Md.).

Cells were grown as monolayers in RPMI medium supplemented with 10% fetal calf serum (InVitrogen, Cergy-Pontoise, France), 2 mM glutamine, 100 units/ml penicillin and 100 mg/ml streptomycin. All cells were split twice a week using trypsin/EDTA (0.25% and 0.02%, respectively; InVitrogen, Cergy-Pontoise, France) and seeded at a concentration of 2.5×10⁴ cells/ml. All cell lines were tested regularly for Mycoplasma contamination by PCR using a Stratagene kit (La Jolla, Calif.).

The combinations were tested either by simultaneous or sequential exposure of the tumor cell lines to PM02734 and the other drug:

a. For simultaneous drug exposure, cells were seeded at 2×10³ cells/well in 96-well plates and treated 24 hours later with the two drugs at the same time either with increasing concentrations of PM02734 trifluoroacetate salt alone or the other drug in various concentrations corresponding to the IC₂₀, IC₄₀ or IC₆₀ values. After approximately four doubling times (120 hours), the growth inhibitory effect was measured by the MIT assay. Combination studies were performed in comparison to single drug incubation for each compound.

b. For sequential drug exposure, cells were seeded at 2×10³ cells/well in 96-well plates and allowed to grow for 24 hours. Cells were then exposed to various concentrations of the first drug for 24 hours (48 hours), the drug was removed, the cells were washed and the second drug was added. After an additional drug exposure, the second drug was removed, the cells were washed and post-incubated in drug-free medium for 72 hours. Growth inhibition was then determined by the MIT assay.

The MIT assay was carried out as described previously (Hansen et al. J. Immunol. Methods, 1989, 119(2), 203-210). In brief, cells were seeded in 96-well tissue culture plates at a density of 2×10³ cells/well. Cell viability was determined after 120 hours incubation by the colorimetric conversion of yellow, water-soluble tetrazolium MIT (3-[4,5-dimethylthiazol-2-yl]-2,5-diphenyltetrazolium bromide; Sigma, Saint-Quentin Fallavier, France), into purple, water-insoluble formazan. This reaction is catalyzed by mitochondrial dehydrogenases and is used to estimate the relative number of viable cells (Mosmann, J. Immunol. Methods, 1983, 65(1-2), 55-63). Cells were incubated with 0.4 mg/ml MTT for 4 hours at 37° C. After incubation, the supernatant was discarded, the cell pellet was resuspended in 0.1 ml of DMSO and the absorbance was measured at 560 nm by use of a microplate reader (Molecular Devices, Menlo Park, Calif.). Wells with untreated cells or with drug-containing medium without cells were used as positive and negative controls respectively. Growth inhibition curves were plotted as a percentage of untreated control cells.

Effects of drug combinations were evaluated using the Chou and Talalay method which is based on the median-effect principle (Chou and Talalay, Adv. Enzyme Regul. 1984, 22, 27-55). This involves plotting dose-effect curves for each drug and for multiple diluted, fixed-ratio combinations, using the equation: fₐ/fᵤ=(C/Cₘ)^{m}, where C is the drug concentration, ICₘ the concentration required for a half-maximal effect (i.e., IC₅₀=50% inhibition of cell growth), fₐ the cell fraction affected by the drug concentration C (e.g., 0.9 if cell growth is inhibited by 90%), fᵤ the unaffected fraction, and m the sigmoidicity coefficient of the concentration-effect curve. On the basis of the slope of the curve for each drug in a combination, it can be determined whether the drugs have mutually nonexclusive effects (e.g., independent or interactive modes of action). The combination index (CI) is then determined by the equation:

CI=[(C)₁/(Cₓ)₁]+[(C)₂/(Cₓ)₂]+[α(C)₁(C)₂/(Cₓ)₁(Cₓ)₂],

where (Cx)₁ is the concentration of drug 1 required to produce an x percent effect of that drug alone, and (C)₁ is the concentration of drug 1 required to produce the same x percent effect in combination with (C)₂. If the mode of action of the drugs is mutually exclusive or nonexclusive, then α is 0 or 1, respectively. CI values were calculated by solving the equation for different values of fₐ (i.e., for different degrees of cell growth inhibition). CI values of <1 indicate synergy, the value of 1 indicates additive effects, and values >1 indicate antagonism. Data were analyzed using concentration-effect analysis CalcuSyn software (Biosoft, Cambridge, UK). For statistical analysis and graphs Prism software

(GraphPad, San Diego, USA) was used. Results are expressed as the mean±standard deviation of at least 3 experiments performed in duplicate.

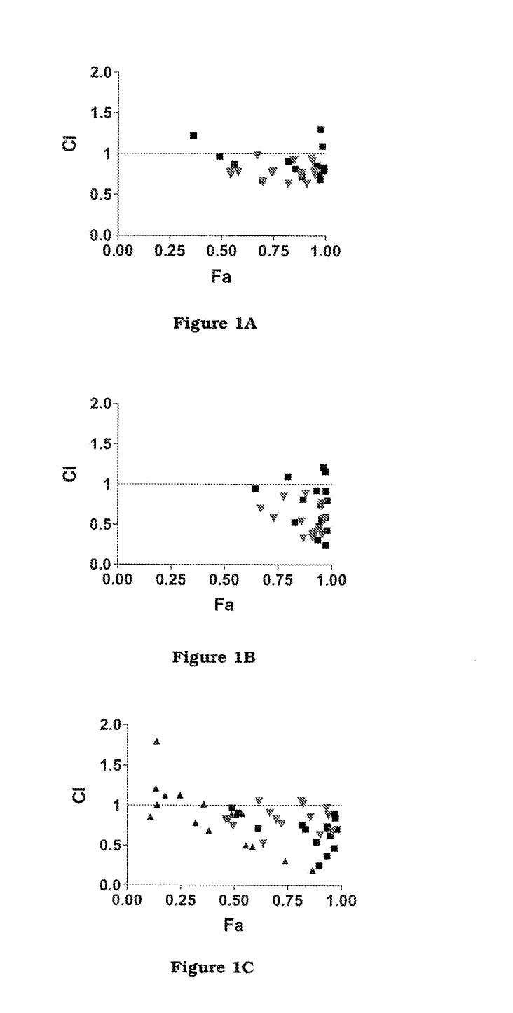

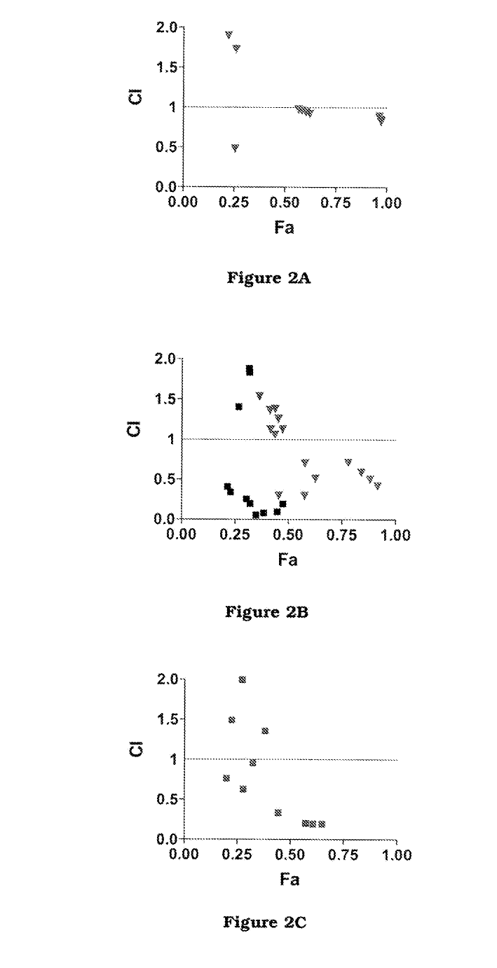

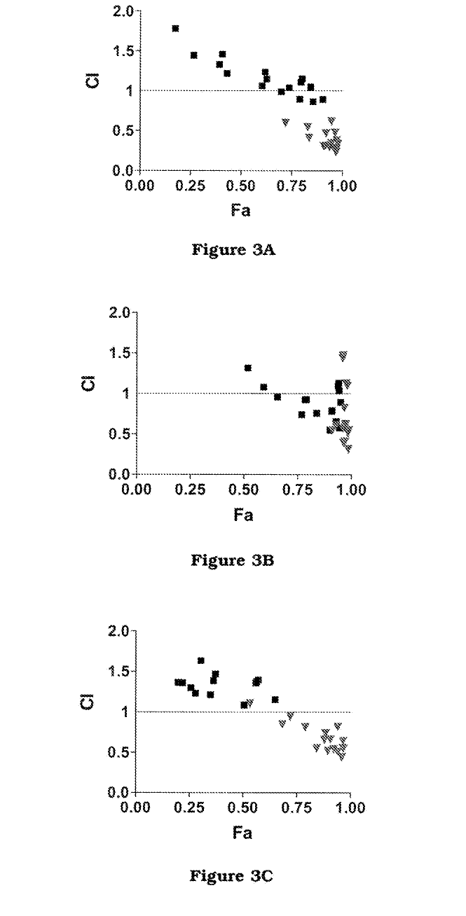

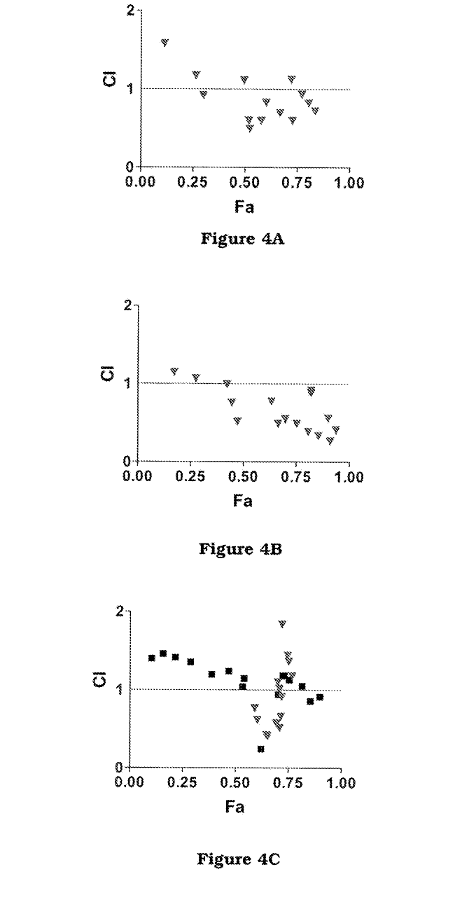

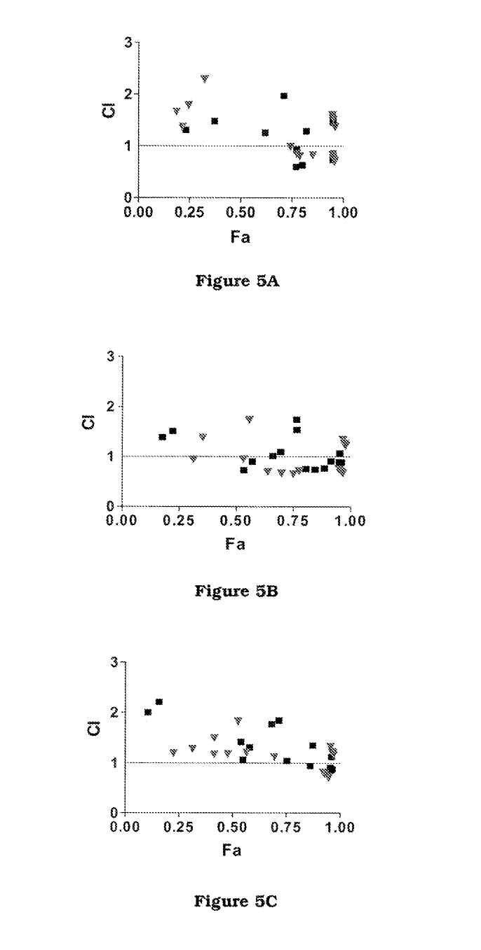

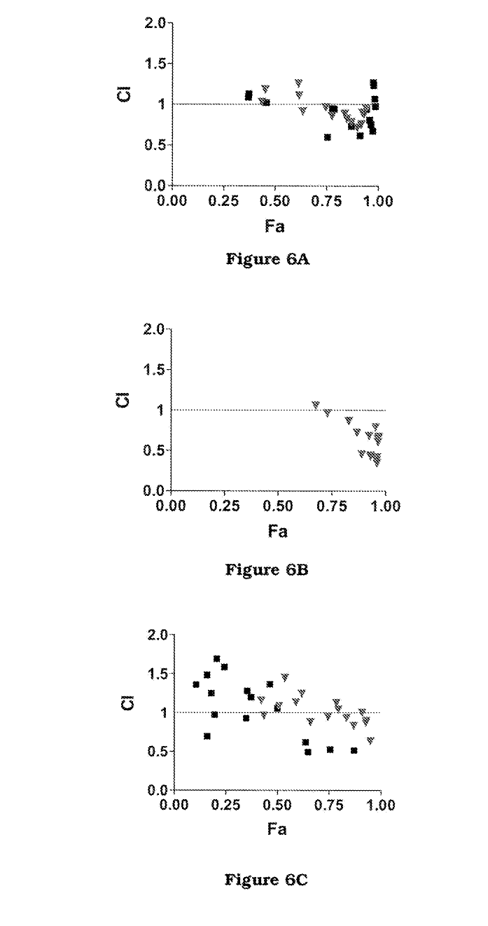

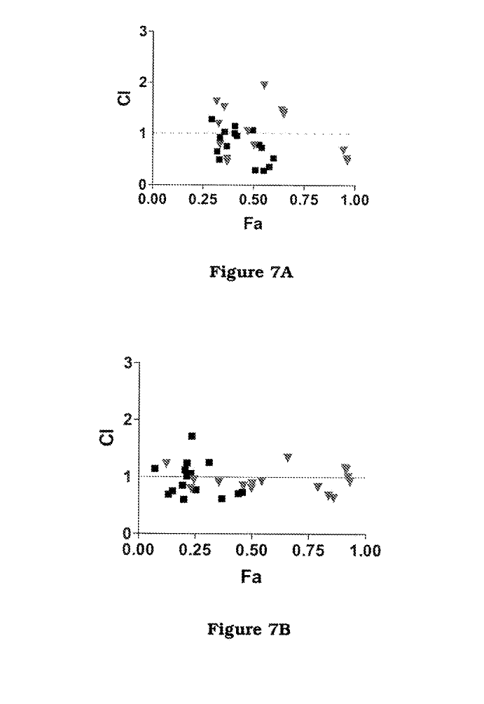

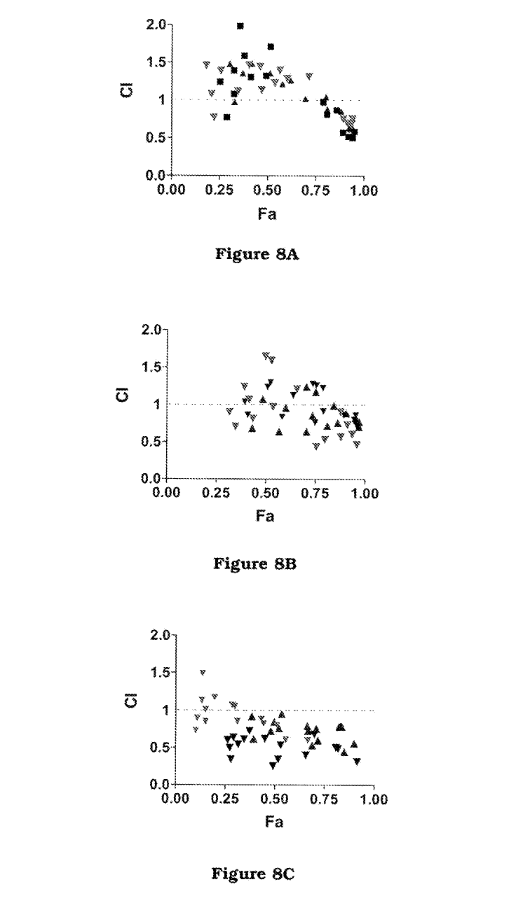

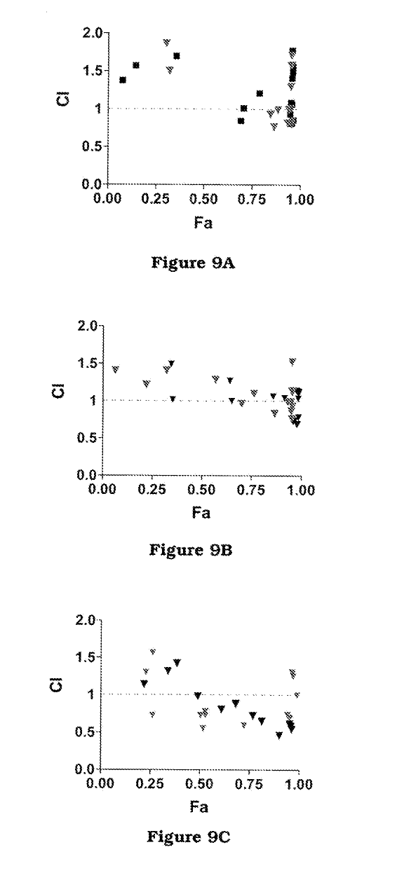

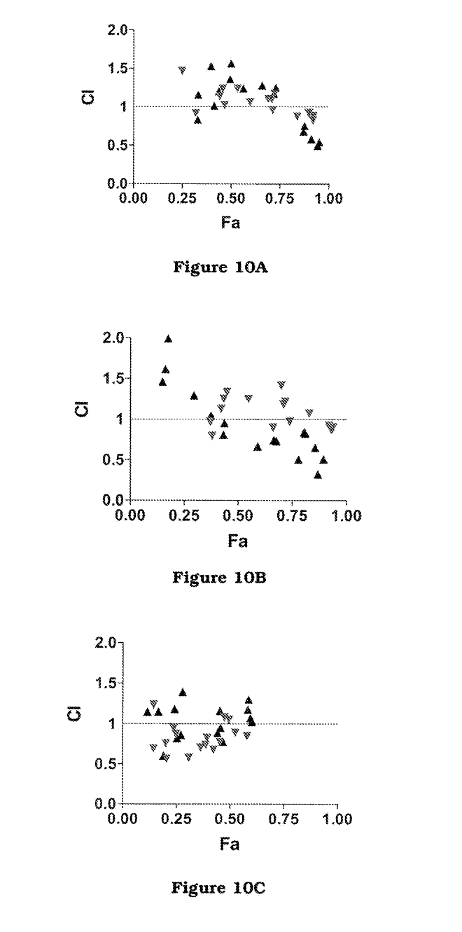

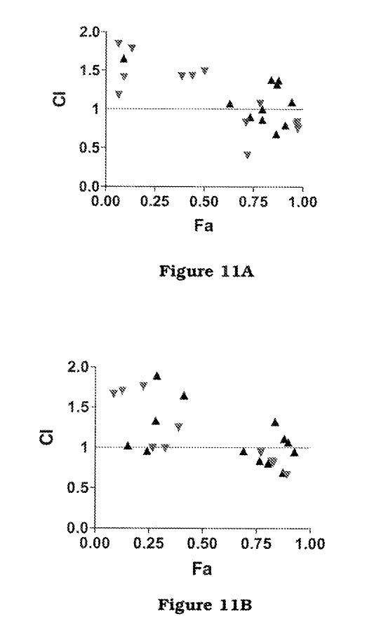

The effect of the tested drug combinations on cell proliferation using different schedules is shown in FIGS. 1-11:
Combination of PM02734 with Oxaliplatin. The combination of PM02734 with Oxaliplatin in DU145 (FIG. 1) and Colo205 (FIG. 2) cell lines resulted in synergistic effects (CI<1) irrespective of concentrations used. The effects of PM02734 in combination with Oxaliplatin did not appear to be schedule dependent.
Combination of PM02734 with Cisplatin. The combination of PM02734 with Cisplatin in DU145 cell line (FIG. 3) resulted in some synergistic effects (CI<1) at high concentrations of both drugs. The effects of PM02734 in combination with Cisplatin did not appear to be schedule dependent.
Combination of PM02734 with 5-FU. The combination of PM02734 with 5-FU in DU145 (FIG. 4) and Colo205 (FIG. 5) cell lines resulted in synergistic effects (CI<1) when PM02734 was added after 5-FU. When PM02734 was administered prior to or simultaneously with 5-FU the effects were additive/synergistic in DU145 and antagonistic in Colo205 cells.
Combination of PM02734 with Gemcitabine. The combination of PM02734 with Gemcitabine in DU145 cell line (FIG. 6) resulted in some synergistic effects (CI<1) at high concentrations when PM02734 was added after Gemcitabine.
Combination of PM02734 with Trabectedin. The combination of PM02734 with Trabectedin in DU145 cell line (FIG. 7) resulted in some synergistic effects (CI<1) when PM02734 was given after or simultaneously with Trabectedin.
Combination of PM02734 with Rapamycin. The combination of PM02734 with Rapamycin in DU145 (FIG. 8) and Colo205 (FIG. 9) cell lines resulted in synergistic effects (CI<1) when PM02734 was added simultaneously with Rapamycin. The effects were additive when PM02734 was administered after Rapamycin and mostly antagonistic for the opposite sequence.
Combination of PM02734 with Sunitinib. The combination of PM02734 with Sunitinib in DU145 (FIG. 10) and Colo205 (FIG. 11) cell lines was at least additive resulting in some synergistic effects (CI<1).

1. A method of treating cancer comprising administering to a patient in need of such treatment a therapeutically effective amount of PM02734, or a pharmaceutically acceptable salt thereof, and a therapeutically effective amount of another anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof.

2. A method of increasing the therapeutic efficacy of an anticancer drug selected from Cisplatin, Gemcitabine, Paclitaxel, Oxaliplatin, 5-Fluorouracil, Trahectedin, Rapamycin, and Sunitinib in the treatment of cancer, which comprises administering, to a patient in need thereof, said anticancer drug and a therapeutically effective amount of PM02734, or a pharmaceutically acceptable salt thereof.

3-20. (canceled)

21. The method according to claim 1, wherein the cancer to be treated is selected from testicular cancer, bladder cancer, lung cancer, gullet cancer, stomach cancer, ovarian cancer, breast cancer, pancreatic cancer, colorectal cancer, leukemia, melanoma, and prostate cancer.

22. The method according to claim 2, wherein the cancer to be treated is selected from testicular cancer, bladder cancer, lung cancer, gullet cancer, stomach cancer, ovarian cancer, breast cancer, pancreatic cancer, colorectal cancer, leukemia, melanoma, and prostate cancer.

23. The method accordin.g to claim 21, wherein PM02734, or a pharmaceutically acceptable salt thereof, and the other anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, form part of the same composition.

24. The method according to claim 21, wherein PM02734, or a pharmaceutically acceptable salt thereof and the other anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, are provided as separate compositions for administration at the same time or at different times.

25. The method according to claim 24, wherein PM02734, or a pharmaceutically acceptable salt thereof and the other anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof are provided as separate compositions for administration at different times.

26. The method according to claim 25, wherein the anticancer drug combined with PM02734 is Cisplatin or a pharmaceutically acceptable salt thereof.

27. The method according to claim 25, wherein the anticancer drug combined with PM02734 is Gemcitabine or a pharmaceutically acceptable salt thereof.

28. The method according to claim 25, wherein the anticancer drug combined with PM02734 is Paclitaxel or a pharmaceutically acceptable salt thereof.

29. The method according to claim 25, wherein the anticancer drug combined with PM02734 is Oxaliplatin or a pharmaceutically acceptable salt thereof.

30. The method according to claim 25, wherein the anticancer drug combined with PM02734 is 5-Fluorouracil or a pharmaceutically acceptable salt thereof.

31. The method according to claim 25, wherein the anticancer drug combined with PM02734 is Trabectedin or a pharmaceutically acceptable salt thereof.

32. The method according to claim 25, wherein the anticancer drug combined with PM02734 is Rapamycin or a pharmaceutically acceptable salt thereof.

33. The method according to claim 25, wherein the anticancer drug combined with PM02734 is Sunitinib or a pharmaceutically acceptable salt thereof.

34. The method according to claim 22, wherein PM02734, or a pharmaceutically acceptable salt thereof, and the other anticancer drug selected from Cisplatin, Gemcitabine, Paclitaxel, Oxaliplatin, 5-Fluorouracil, Trabectedin, Rapamycin, and Sunitinib, or pharmaceutically acceptable salt thereof, form part of the same composition.

35. The method according to claim 22, wherein PM02734, or a pharmaceutically acceptable salt thereof. and the other anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, are provided as separate compositions for administration at the same time or at different times.

36. The method according to claim 22, wherein PM02734, or a pharmaceutically acceptable salt thereof, and the other anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, are provided as separate compositions for administration at different times.

37. A kit for administering PM02734, or a pharmaceutically acceptable salt thereof, in combination with another anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, comprising a dosage form of PM02734, or a pharmaceutically acceptable salt thereof, and/or a dosage form of the other anticancer drug selected from Cisplatin, Gemcitabine, Paclitaxel, Oxaliplatin, 5-Fluorouracil, Trabectedin, Rapamycin, and Sunitinib, or a pharmaceutically acceptable salt thereof, and printed instructions for administering both drags in combination.
